(19) United States
(12) Patent Application Publication
(10) Pub. No.: US 2013/0018020 A1
(43) Pub. Date: Jan. 17, 2013

(54) INHIBITOR FOR MELANIN, AND COSMETIC COMPOSITION CONTAINING SAME

(71) Applicants: Hong Ju Shin, Seongnam-si (KR); Do Hoon Kim, Seoul (KR); Jeong Hwan Kim, Paju-si (KR); Hyun Ju Koh, Anyang-si (KR); Won Seok Park, Seoul (KR)

(72) Inventors: Hong Ju Shin, Seongnam-si (KR); Do Hoon Kim, Seoul (KR); Jeong Hwan Kim, Paju-si (KR); Hyun Ju Koh, Anyang-si (KR); Won Seok Park, Seoul (KR)

(73) Assignee: AMOREPACIFIC CORPORATION, Seoul (KR)

(21) Appl. No.: 13/638,643

(22) PCT Filed: Mar. 30, 2011
(86) PCT No.: PCT/KR11/02170

§ 371 Date: Oct. 1, 2012

(30) Foreign Application Priority Data
Mar. 31, 2010 (KR) 1020100029221

(51) Int. Cl. A61K 31/455; A61K 31/60; A61P 17/00

(52) U.S. Cl. 514/161; 514/355

(57) ABSTRACT

The present invention relates to a melanin production inhibitor containing, as active ingredients, tranexamic acid and nicotinic acid amide, which inhibits the formation of skin melanocytes, and to a cosmetic composition which contains the melanin production inhibitor as an active ingredient and has the effects of reducing liver spots, blemishes, freckles and post-inflammatory hyperpigmentation, improving skin tone and texture and whitening the skin.

### TECHNICAL FIELD

The present invention relates to a melanin production inhibitor containing, as active ingredients, tranexamic acid and nicotinic acid amide, which inhibits the formation of skin melanocytes, and to a cosmetic composition which contains the melanin production inhibitor as an active ingredient and has the effects of reducing liver spots, blemishes, freckles and post-inflammatory hyperpigmentation, improving skin tone and texture and whitening the skin.

### BACKGROUND ART

The skin has physical and chemical factors, which defend against UV rays, and has the property of minimizing skin damage caused by various photochemical reactions. The horny layer reflects and diffuses UV rays to attenuate the energy of UV rays. In addition, melanin, SOD (superoxide dismutase) and other antioxidant components absorb UV rays that penetrated the skin, and attenuate the energy of UV rays, or eliminate reactive oxygen species caused by UV rays, thereby preventing skin damage. However, if the body is exposed to a large amount of UV rays exceeding the ability of the above defense factors or if the ability of the defense factors is reduced due to aging, various skin disorders occur.

The skin has various immune cells, including macrophages and Langerhans cells. UV radiation reduces not only the number of such cells, but also the function. The skin color is mainly determined by the distribution and amount of melanin in the skin. Melanin is produced in melanocytes in which enzymes such as tyrosinase are present. These enzymes form the dark-brown pigment melanin by an oxidation reaction in which the amino acid tyrosine present in the body is polymerized into a matrix.

The formed melanin migrates to epidermal keratinocytes through the dendrites of melanocytes. In the keratinocytes, the melanin forms a hat-like structure around the nucleus to protect genes from UV rays and remove free radicals to protect intracellular proteins.

In the body, there is no enzyme that degrades melanin. Melanin is removed from the skin when keratinocytes are detached from the epidermis. If melanin is produced more than required, it causes hyperpigmentations such as liver spots, freckles or spots, which are unfavorable in terms of beauty.

Various factors having effects on melanin production are known, and the stimulation of melanin production by UV rays and the resulting pigmentation are important in the cosmetic field. The fundamental functions of drugs which are added to whitening cosmetic products in order to prevent pigmentation include the inhibition of tyrosinase activity, the inhibition of tyrosinase production, the inhibition of a mediator of melanin production, the inhibition of reduction and photooxidation of existing melanin, the promotion of melanin excretion, and the blocking of UV rays.

With the desire of women to have a white skin even in an environment in which they are exposed to UV rays, a demand for preventing and ameliorating skin pigment abnormalities and hyperpigmentations has increased. Thus, the development of whitening products for preventing the excessive production of melanin has been required, and many efforts have been made to develop such whitening products. For example, tyrosinase activity inhibitors, such as kojic acid and arbutin, hydroquinone, vitamin A, vitamin C, and derivatives thereof, have been used in whitening products. However, the use thereof has been limited due to problems associated with safety for the skin and stability in formulations, and insufficient whitening effects.

### DISCLOSURE

### Technical Problem

Accordingly, the present inventors have found that, when tranexamic acid and nicotinic acid amide are used in combination with each other, they can inhibit the formation of skin melanocytes, prevent or ameliorate skin pigmentations such as blemishes, freckles and liver spots, and relieve skin irritation, thereby completing the present invention. In addition, the present inventors have found that, when a composition contains hydroxyl acid in addition to tranexamic acid and nicotinic acid amide, the whitening effect thereof is further improved.

Therefore, it is an object of the present invention to provide a melanin production inhibitor for inhibiting the formation of skin melanocytes.

Another object of the present invention is to provide a cosmetic composition containing the melanin production inhibitor.

### Technical Solution

In order to accomplish the above objects, the present invention provides a melanin production inhibitor containing tranexamic acid and nicotinic acid amide as active ingredients.

The present invention also provides a cosmetic composition which contains the melanin production inhibitor as an active ingredient and has the effects of reducing liver spots, blemishes, freckles and post-inflammatory hyperpigmentation, improving skin tone and texture and whitening the skin.

### Advantageous Effects

A melanin production inhibitor according to the present invention effectively inhibits the formation of skin melanocytes, and a cosmetic composition containing the melanin production inhibitor as an active ingredient can prevent or ameliorate skin pigmentations such as liver spots, blemishes and freckles and improve overall skin tone to make the skin clean and to whiten the skin. In addition, the cosmetic composition can minimize skin irritation to ensure skin safety together with an excellent whitening effect.

### BEST MODE

The present invention relates to a melanin production inhibitor containing tranexamic acid and nicotinic acid amide as active ingredients.

Generally, tranexamic acid serves as a hemostatic agent when it is administered orally, and serves as a blood coagulating agent or an anti-inflammatory agent when it is applied to the skin. In the present invention, tranexamic acid is used to inhibit melanin production.

Nicotinic acid amide is also known as pyridine-3-carboxamide, nicotinamide or niacinamide. It has a molecular weight of C₆H₆N₂O. It is a vitamin B complex, like nicotinic acid, and is known as an anti-pellagra factor in a human, an anti-black tongue factor in a dog and a microbial growth factor. It coexists with nicotinic acid in plants and is widely distributed. In a living body, nicotinic acid amide is present as nicotinic acid amide nucleotide (NAD⁺ or NADP⁺) which is a coenzyme type of this vitamin. In addition, it is a coenzyme of dehydrogenase, which is involved in many oxidation/reduction reactions.

Each of tranexamic acid and nicotinic acid amide is contained in an amount of 0.001-10.0 wt %, and preferably 3.0-5.0 wt %, based on the total amount of the melanin production inhibitor according to the present invention. If the content of each of the active ingredients is less than 0.001 wt %, the effect thereof will be insignificant, and if the content is more than 10 wt %, an increase in the content will not lead to a significant increase in the effect and can cause skin irritation.

In addition, the melanin production inhibitor according to the present invention may comprise, in addition to the above active ingredients, α- and β-hydroxy acids which are mixed with each other at a weight ratio of 0.2:1 to 30:1, and preferably 2:1 to 10:1.

α-Hydroxy acid that is used in the present invention is a colorless and odorless crystal structure, dissolves well in water and is extracted from sugar canes and fruits such as oranges. It has a small and simple molecular structure, and thus penetrates the skin deeply. It has an excellent effect of removing keratin by weakening keratin lipids. In addition, it improves skin texture and inhibits the production of melanocytes, thereby reducing pigmentations and living spots, which are caused by skin aging, and increasing skin elasticity.

Examples of α-hydroxy acid that is used in the present invention include glycolic acid, lactic acid, malic acid, citric acid, and tartaric acid. Among them, glycolic acid (OHCH₂COOH) is preferably used. In the present invention, α-hydroxy acid is preferably contained in an amount of 0.0002-15 wt % based on the total weight of the melanin production inhibitor. If the content of α-hydroxy acid is more than 15 wt %, it will increase skin irritation, and if the content is less than 0.0002 wt %, the effect thereof will be insignificant.

β-hydroxy acid that is used in the present invention refers to a collection of substances having a structure in which an alcohol or hydroxyl group is attached to the carbon atom at the β-position of carboxyl acid. It acts on the epidermis to stimulate the turn-over of the horny layer, thereby thinning the color of produced melanin. In dermatology, it is used in skin scaling or peeling. Examples of this β-hydroxy acid include β-hydroxybutyric acid, β-hydroxy β-methylbutyrate, carnitine, and β-hydroxypropionic acid. The most typical β-hydroxy acid is salicylic acid represented by the following formula 1.

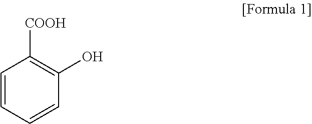

In the present invention, β-hydroxy acid is contained in an amount of 0.001-5.0 wt % based on the total weight of the melanin production inhibitor. If the content of β-hydroxy acid is more than 5.0 wt %, it will increase skin irritation, and if the content is less than 0.001 wt %, the effect thereof will be insignificant.

The present invention also relates to a cosmetic composition containing the melanin production inhibitor. The cosmetic composition contains, as an active ingredient, the melanin production inhibitor which has the effects of reducing liver spots, blemishes, freckles and post-inflammatory hyperpigmentation, improving skin tone and texture and whitening the skin. The cosmetic composition according to the present invention may contain the melanin production inhibitor in an amount of 0.001-15.0 wt % based on the total weight of the composition. If the content of the melanin production inhibitor in the cosmetic composition is less than 0.001 wt %, it will not show a significant effect, and if the content is more than 15.0 wt %, an increase in the content will not lead to a significant increase in the effect and can cause skin irritation.

In one embodiment of the present invention, the cosmetic composition can be formulated as a beauty aid composition. This beauty aid composition may be, for example, a cosmetic product. In this case, the cosmetic composition according to the present invention contains a cosmetically or dermatologically acceptable medium or base. It may be prepared into any topically applicable formulation, for example, a solution, a gel, a solid, an anhydrous paste, an oil-in-water emulsion, a suspension, a micro-emulsion, a microcapsule, a microgranule, an ionic (liposome) or nonionic vesicular dispersion, a cream, a skin softener, a lotion, a powder, an ointment, a spray or a conceal stick. The composition may be prepared according to a conventional method known in the art. The composition according to the present invention may be used in the form of foam or an aerosol composition further including a pressurized propellant.

In addition, the cosmetic composition according to the present invention may comprise an adjuvant commonly used in the field of cosmetology or dermatology, such as a fat material, an organic solvent, a solubilizer, a thickener, a gelling agent, a softener, an antioxidant, a suspending agent, a stabilizer, a foaming agent, an aromatic agent, a surfactant, water, an ionic or nonionic emulsifier, a filler, a sequestering agent, a chelating agent, a preservative, vitamin, a blocking agent, a wetting agent, essential oil, a dye, a pigment, a hydrophilic or lipophilic agent, a lipid vesicle or any other ingredients commonly used in cosmetics. The adjuvant may be contained in an amount generally used in the field of cosmetology or dermatology.

The formulation of the cosmetic composition according to the present invention is not particularly limited and may be selected appropriately depending on the desired purpose. For example, the cosmetic composition may be prepared as a formulation such as a skin softener (skin lotion or milk lotion), nourishing lotion, essence, nourishing cream, massage cream, eye cream, eye essence, a pack, a patch, gel, a stick, a spray, cleansing cream, cleansing foam, cleansing water, pack, powder, body lotion, body cream, body oil or body essence, but is not limited thereto.

In one embodiment of the present invention, the cosmetic composition may be applied to a face, particularly the eye rim, the mouth, the cheek or the forehead, a neck, hands, feet or the like, but is not limited thereto.

### MODE FOR INVENTION

Hereinafter, the present invention will be described in further detail with reference to examples and test examples. It is to be understood, however, that the scope of the present invention is not limited by these examples and that the modifications, substitutions and additions generally known in the art may be made within the scope of the present invention.

### Test Example 1

### Effect on Inhibition of Melanin Production

Human melanoma HM3KO cells (Y. Funasaka, Department of dermatology, Kobe university school of medicine, 5-1 Kusunoki-cho 7-chrome, Chuo-ku, Kobe 650, Japan) were cultured in 10% FBS-containing MEM (Minimum Essential Medium) under the conditions of 37° C. and 5% CO₂. The cultured cells were plated on T75 flasks at a density of 3×10⁵ cells per flask and allowed to stand overnight until the cells adhered to the flask wall. After the cells were confirmed to adhere to the flask wall, the medium was replaced with a fresh medium containing 10 ppm of each of the test materials shown in Table 1 below. In a control group, a DMSO-containing medium was used. In this manner, the medium was replaced with a fresh medium containing each test material once at intervals of 2-3 days, while the cells were cultured until the flask was filled with the cells. When the cells completely grew, the cells were collected, and the color thereof was compared between the control group and each of the groups treated with the test material. In addition, after the culture medium has been removed and the cells were washed with PBS, the cells were dissolved in 1N sodium hydroxide, and the absorbance at 500 nm was measured. Based on the measurements, the inhibition (%) of melanin production was calculated using the following equation 1, and the results of the calculation are shown in Table 1 below.

Inhibition (%) of melanin production=100×(absorbance of each test material/absorbance of control) [Equation 1]

TABLE 1

| Test material | Inhibition (%) of melanin production |
|---|---|
| Kojic acid | 28.3 |
| Tranexamic acid | 32.2 |
| Nicotinic acid amide | 29.5 |
| Tranexamic acid + nicotinic acid | 56.7 |
| amide | |

As can be seen in Table 1 above, the effects of tranexamic acid and nicotinic acid amide on the inhibition of melanin production were excellent when they were used in combination with each other compared to when they were used alone.

### Examples 1 and 2 and Comparative Examples 1 to 4

According to the components and contents shown in Table 2 below, cosmetic compositions of Examples 1 and 2 and Comparative Examples 1 to 4 were prepared. Specifically, components 1 to 7 were mixed with each other and dissolved at 70° C. to obtain an aqueous phase. Meanwhile, components 10 to 16 were dissolved at 70° C. to obtain an oily phase. The oily phase was added to the aqueous phase, and the mixture was stirred with a homomixer (Tokushu Kika, Japan) to prepare an emulsion. Then, components 17 and 18 were added to the emulsion to make the emulsion fragrant and thick. The mixture was defoamed and then cooled to room temperature, thereby preparing cosmetic compositions of Examples 1 and 2 and Comparative Examples 1 to 4.

TABLE 2
(unit: wt %)

| | | | Comparative | Comparative | Comparative | Comparative |
|---|---|---|---|---|---|---|
| Component | Example 1 | Example 2 | Example 1 | Example 2 | Example 3 | Example 4 |
| 1. purified water | Balance | Balance | Balance | Balance | Balance | Balance |
| 2. glycerin | 3.0 | 3.0 | 3.0 | 3.0 | 3.0 | 3.0 |
| 3. butylene glycol | 5.0 | 5.0 | 5.0 | 5.0 | 5.0 | 5.0 |
| 4. beta-glucan | 5.0 | 5.0 | 5.0 | 5.0 | 5.0 | 5.0 |
| 5. hyaluronic acid | 3.0 | 3.0 | 3.0 | 3.0 | 3.0 | 3.0 |
| extract | | | | | | |
| 6. carbomer | 0.1 | 0.1 | 0.1 | 0.1 | 0.1 | 0.1 |
| 7. tranexamic acid | 1.0 | 1.0 | 2.0 | — | 2.0 | — |
| 8. nicotinic acid | 1.0 | 1.0 | — | 2.0 | — | 2.0 |
| amide | | | | | | |
| 9. glycolic acid | — | 5.0 | — | — | 5.0 | 5.0 |
| 10. caprylic/capric | 8.0 | 8.0 | 8.0 | 8.0 | 8.0 | 8.0 |
| triglyceride | | | | | | |
| 11. hydrogenated | 5.0 | 5.0 | 5.0 | 5.0 | 5.0 | 5.0 |
| polydecene | | | | | | |
| 12. cetearyl | 1.5 | 1.5 | 1.5 | 1.5 | 1.5 | 1.5 |
| glucoside | | | | | | |
| 13. sorbitan stearate | 0.4 | 0.4 | 0.4 | 0.4 | 0.4 | 0.4 |
| 14. cetearyl alcohol | 1.0 | 1.0 | 1.0 | 1.0 | 1.0 | 1.0 |
| 15. salicylic acid | — | 0.4 | — | — | 0.4 | 0.4 |
| 16. preservative | q.s. | q.s. | q.s. | q.s. | q.s. | q.s. |
| 17. fragrance | q.s. | q.s. | q.s. | q.s. | q.s. | q.s. |
| 18. triethanolamine | 0.1 | 0.1 | 0.1 | 0.1 | 0.1 | 0.1 |

### Test Example 2

### Whitening Effect on Human Skin

In order to examine the whitening effect and skin irritation of the compositions on the human skin, the following test was carried out on 30 healthy men and women (average age: 35.2). Specifically, an opaque tape having 6 perforations of a 1.5-cm diameter was attached to the upper arm portion of each of the subjects, and then the attached portion was radiated with UVB at a dose about 1.5-2 times the minimal erythema dose of each subject to induce skin darkening. After the UV radiation, each of the compositions was applied on the UV-radiated portion, and after 2 months, the color of the skin was measured using a colorimeter. The cosmetic compositions of Examples 1 and 2 and Comparative Examples 1 to 4 were applied to each of the subjects twice (morning and evening) a day everyday.

The judgment of the effect of each composition was performed by determining the “L” value that indicates the lightness and darkness of the skin. For reference, the non-sunburned skins of Korean people generally show an “L” value of 50-70. The effect of each composition was judged by measuring the color of the skin using the colorimeter CR2002 (Japan, Minolta). Although the L*a*b* color system is generally used to indicate colors, the L* value (lightness) was mainly used as an index in the present invention. The L* value was corrected with a standard plate, and the measurement was repeated five times or more on one portion so that the pigmented portions were uniformly measured. The difference (ΔL*) in skin color between the time point of initiation of application and the time point of completion of application of each composition was calculated according to the following equation 2, and the calculation results are shown in Table 3 below. Meanwhile, the whitening effect is evaluated by comparing the ΔL* value between the sample-applied portion and the control portion. A ΔL* value of about 2 indicates that pigmentation is clearly relieved, and if it is more than about 1.5, it can be judged to have a whitening effect.

ΔL*=L*value 2 months after the initiation of application−L*value at the time of initiation of application [Equation 2]

When the applied test material is effective, the L value gradually increases. The comparison between the test materials was performed by calculating the difference (ΔL) in skin color between the time point of initiation of application and the time point of completion of application (after 2 months) using equation 2, and the results of the calculation are shown in Table 3 below.

TABLE 3

| Test material | Whitening effect (ΔL) |
|---|---|
| Example 1 | 1.52 |
| Example 2 | 2.43 |
| Comparative Example 1 | 1.03 |
| Comparative Example 2 | 0.96 |
| Comparative Example 3 | 1.95 |
| Comparative Example 4 | 1.82 |

As can be seen from the results in Table 3 above, the whitening effects of the compositions of Examples 1 and 2 in which tranexamic acid nicotinic acid amide were used in combination with each other were significantly excellent compared to those of Comparative Examples 1 to 4 in which they were used alone. Moreover, the composition of Example 2 containing glycolic acid (α-hydroxy acid) in addition to tranexamic acid and nicotinic acid amide showed an excellent whitening effect compared to the composition of Example 1.

### Test Example 3

### Test for Effect on Reduction in Age Spots on Human Skin

60 men and women (over 50 years old) with age spots were divided into 6 groups, each consisting of 10 persons, and the compositions of Examples 1 and 2 and Comparative Examples 1 to 4 were used in the 6 groups, respectively. Each of the test materials was applied to the age spot portion of each subject in the evening everyday for 2 weeks, after which the effect of each test material on a reduction in the age spots was scored from 1 (no effect) to 7 (the best effect). The average scores of the test materials are shown in Table 4 below.

TABLE 4

| | Age spot-reducing effect |
|---|---|
| Test material | (perfect score: 7) |
| Example 1 | 4.9 |
| Example 2 | 6.8 |
| Comparative Example 1 | 3.9 |
| Comparative Example 2 | 3.4 |
| Comparative Example 3 | 5.8 |
| Comparative Example 4 | 5.1 |

As can be seen from the results in Table 4 above, the age spot-reducing effects of the compositions of Examples 1 and 2 compared to those of Comparative Examples 1 to 4.

### Test Example 4

### Examination of Human Skin Irritation

The compositions of Examples 1 and 2 and Comparative Examples 1 to 4 were applied to the subjects of Test Example 3 for 2 months, after skin irritation was compared between the compositions. The skin irritation of each of the compositions was scored from 1 (no irritation) to 5 (highest irritation) by each of the subjects. The average scores of the compositions are shown in Table 5 below.

TABLE 5

| Test material | Skin irritation |
|---|---|
| Example 1 | 1.63 |
| Example 2 | 1.95 |
| Comparative Example 1 | 1.14 |
| Comparative Example 2 | 1.86 |
| Comparative Example 3 | 3.17 |
| Comparative Example 4 | 2.77 |

As can be seen from the results in Table 5 above, the cosmetic compositions of Examples 1 and 2 according to the present invention had excellent whitening effects and less skin irritation compared to the compositions of Comparative Examples 1 to 4. Particularly, the cosmetic composition of Example 2 showed the best whitening effect while the skin irritation thereof was significantly low.

1. A melanin production inhibitor containing tranexamic acid and nicotinic acid amide as active ingredients.

2. The melanin production inhibitor of claim 1, wherein each of the tranexamic acid and the nicotinic acid amide is contained in an amount of 0.001-10 wt % based on the total weight of the melanin production inhibitor.

3. The melanin production inhibitor of claim 1, further containing one or more selected from the group consisting of α- and β-hydroxy acids.

4. The melanin production inhibitor of claim 3, wherein the α- and β-hydroxy acids are contained at a weight ratio of 0.2:1-30:1 relative to the melanin production inhibitor.

5. The melanin production inhibitor of claim 3, wherein the α-hydroxy acid is one or more selected from the group consisting of glycolic acid, lactic acid, malic acid, citric acid and tartaric acid.

6. The melanin production inhibitor of claim 3, wherein the β-hydroxy acid is one or more selected from the group consisting of β-hydroxybutyric acid, β-hydroxy β-methylbutyrate, carnitine, 3-hydroxypropionic acid, and salicylic acid.

7. A cosmetic composition containing, as an active ingredient, a melanin production inhibitor according to claim 1.

8. Use of the composition of claim 7 for reducing liver spots, blemishes, freckles and post-inflammatory hyperpigmentation, improving skin tone and texture or whitening the skin.
